(19) United States
(12) Patent Application Publication
(10) Pub. No.: US 2021/0403904 A1
(43) Pub. Date: Dec. 30, 2021

(54) METHODS FOR HAPLOTYPING WITH SHORT READ SEQUENCE TECHNOLOGY

(71) Applicant: 13.8, Inc., Palo Alto, CA (US)

(72) Inventor: Christina FAN, San Jose, CA (US)

(21) Appl. No.: 17/206,701

(22) Filed: Mar. 19, 2021

Related U.S. Application Data
(63) Continuation of application No. PCT/US2019/052273, filed on Sep. 20, 2019.
(60) Provisional application No. 62/734,047, filed on Sep. 20, 2018.

(51) Int. Cl. C12N 15/10; C12Q 1/6806

(52) U.S. Cl. CPC C12N 15/1082; C12Q 1/6806

(57) ABSTRACT

Provided herein are compositions and methods for preserving proximity data in nucleic acid samples, by embedding indexing information in the samples prior to fragmentation. Further provided herein are transposon libraries for generating such indexed nucleic acid samples.

### CROSS-REFERENCE

This application is a continuation of PCT/US2019/52273, filed Sep. 20, 2019, which claims priority to and the benefit of U.S. Provisional Application No. 62/734,047, filed Sep. 20, 2018, each of which is entirely incorporated herein by reference.

### SEQUENCE LISTING

The instant application contains a Sequence Listing which has been submitted electronically in ASCII format and is hereby incorporated by reference in its entirety. The ASCII copy, created on Oct. 2, 2019, is named 51745-703_601_SL.txt and is 965 bytes in size.

### BACKGROUND

Advances in Next-generation sequencing (NGS) techniques have led to substantial increases in the amount of genomic data available for diagnostics, basic research and other industrial and health-related fields. However, assembly and processing the acquired data into meaningful, accurate scaffolds is in some cases hampered by short read sequences. As such, methods to aid genome assembly are needed.

### SUMMARY

In one aspect, a method of assembling a nucleic acid sequence for a sample nucleic acid comprising: a) contacting the sample nucleic acid to a transposon library comprising: at least 1000 transposon nucleic acid molecules, said at least 1000 transposon nucleic acid molecules having, in order, a first region comprising a transposase binding border common to the transposon library; a second region of at least 5 bases, said second region varying among said at least 1000 transposon nucleic acid molecules; a third region common to the transposon library, wherein the third region has a length sufficient for annealing and extension of a nucleic acid primer; a fourth region of at least 5 bases, said fourth region varying among said at least 1000 transposon nucleic acid molecules; and a fifth region comprising a transpose binding border common to the transposon library, wherein for a transposon nucleic acid of the library, the second region and the fourth region comprise nucleic acids having sequences such that determining a second region sequence and determining a fourth region sequence allows one to assign a sequence read comprising second region sequence and a sequence read comprising fourth region sequence to a common molecule; b) fragmenting the transposon-contacted nucleic acid sample to form transposon-sample chimeric nucleic acids; c) sequencing at least a portion of at least some of the chimeric nucleic acids; and d) assigning chimeric nucleic acid reads sharing a second nucleic acid segment and a fourth nucleic acid indicative of a common origin to a common phase of a sample nucleic acid scaffold. In some embodiments, steps a and b are conducted in a single tube. In some embodiments, the sample nucleic acid originates from a diploid organism. In some embodiments, the sample nucleic acid originates from a polyploid organism. In some embodiments, the common scaffold comprises at least one single nucleotide polymorphism. In some embodiments, generating nucleic acid fragments comprises annealing at least one primer to at least one third region of at least one transposon nucleic acid inserted into a sample nucleic acid molecule, contacting a polymerase with the primer-transposon nucleic acid inserted sample nucleic acid molecule, and extending the primer using the transposon nucleic acid inserted sample nucleic acid molecule as a template. In some embodiments, generating nucleic acid fragments comprises contacting the sample nucleic acid to a sequence specific endonuclease to generate nucleic acid fragments. In some embodiments, the endonuclease is a restriction enzyme, a zinc finger nuclease (ZFN), a transcription activator-like effector nucleases (TALEN), or a CRISPR-Cas9 endonuclease. In some embodiments, generating nucleic acid fragments comprises contacting the sample nucleic acid to a CRISPR-Cas9 nickase, to generate nucleic acid fragments. In some embodiments, assembling the nucleic acid fragments further comprises analysis of paired end read data.

In some aspects, a computer implemented system for generating contigs of nucleic acid sequence information comprises a processor configured to: receive a set of paired end reads; receive indexing data from each paired end read; assign paired end reads to a common phase of a scaffold; and assign commonly indexed reads to a common phase of a scaffold. In some embodiments, the processor is further configured to output processed contigs to a network, screen, or server.

In some aspects, a method for generating contigs of nucleic acid sequence information comprises receiving a set of paired end reads; receiving indexing data from each paired end read; assigning paired end reads to a common phase of a scaffold; and assigning commonly indexed reads to a common phase of a scaffold. In some embodiments, the processor is further configured to output processed contigs to a network, screen, or server.

In some aspects, a nucleic acid transposon comprising in order from the 5′ to 3′ direction: a first region comprising 10-20 bases, and a transposase binding border; a second region comprising 5-10 bases; a third region comprising 20-60 bases, and comprising SEQ ID NO: 1, SEQ ID NO: 2, or SEQ ID NO: 3; a fourth region comprising 5-10 bases; and a fifth region comprising 10-20 bases, and a transposase binding border. In some embodiments, the nucleic acid further comprises at least one transposase. In some embodiments, the transpose is a DDE transposase, tyrosine transposase, serine transposase, rolling circle/Y2 transposase, or reverse transcriptase/endonuclease. In some embodiments, the transpose is Tn5. In some embodiments, the nucleic acid is 60-120 bases in length. In some embodiments, the nucleic acid is about 80 bases in length. In some embodiments, the second region is at least 6 bases in length. In some embodiments, the second region is 6-12 bases in length. In some embodiments, the second region is about 8 bases in length. In some embodiments, the second region sequence indicates the fourth region sequence, such that the second region sequence and the fourth region sequence are identifiable as arising from a common molecule when independently sequenced.

In some aspects, a transposon library comprising: at least 1000 transposon nucleic acid molecules, said at least 1000 transposon nucleic acid molecules having, in order, a first region comprising a transposase binding border common to the transposon library; a second region of at least 5 bases, said second region varying among said at least 1000 transposon nucleic acid molecules; a third region common to the transposon library, wherein the third region has a length sufficient for annealing and extension of a nucleic acid primer; a fourth region of at least 5 bases, said fourth region varying among said at least 1000 transposon nucleic acid molecules; and a fifth region comprising a transposase binding border common to the transposon library, and wherein for a transposon nucleic acid of the library, the second region and the fourth region comprise nucleic acids having sequences such that determining a second region sequence and determining a fourth region sequence allows one to assign a sequence read comprising second region sequence and a sequence read comprising fourth region sequence to a common molecule. In some embodiments, the second region and the fourth region differ by 1 base. In some embodiments, each of the at least 1000 transposons comprises a unique pair of second regions and fourth regions relative to all other members of the library. In some embodiments, each of the at least 1000 transposons comprises at least 5,000 unique pairs of second regions and fourth regions. In some embodiments, each of the at least 1000 transposons comprises at least 10,000 unique pairs of second regions and fourth regions. In some embodiments, each of the at least 1000 transposons comprises at least 50,000 unique pairs of second regions and fourth regions. In some embodiments, each of the at least 1000 transposons comprises a unique second region and a unique fourth region relative to all other members of the library. In some embodiments, each of the at least 1000 transposons comprises a common third region. In some embodiments, each of the at least 1000 transposons comprises a common first region and a common fifth region. In some embodiments, the second region and the fourth region of a transposon differ by less than 2 bases. In some embodiments, the second region and the fourth region of a transposon differ by less than 3 bases. In some embodiments, the transposon library comprises at least 5,000 nucleic acid transposons. In some embodiments, the transposon library comprises at least 10,000 nucleic acid transposons. In some embodiments, the transposon library comprises at least 50,000 nucleic acid transposons.

In some aspects, an indexed sample nucleic acid comprises: a concatamer nucleic acid of sample nucleic acid interrupted by at least one transposon nucleic acid, wherein at least some of the transposon nucleic acid comprises: a first region comprising a transposase binding border common to a transposon library; a second region of at least 5 bases, said second region varying among said at least some transposon nucleic acid molecules; a third region common to the transposon library, wherein the third region has a length sufficient for annealing and extension of a nucleic acid primer; a fourth region of at least 5 bases, said fourth region varying among said at least some transposon nucleic acid molecules; and a fifth region comprising a transposase binding border common to the transposon library, and wherein for the transposon nucleic acid of the library, the second region and the fourth region comprise nucleic acids having sequences such that determining a second region sequence and determining a fourth region sequence allows one to assign a sequence read comprising second region sequence and a sequence read comprising fourth region sequence to a common molecule. In some embodiments, the nucleic acid comprises at least 5 transposons. In some embodiments, the nucleic acid comprises at least 50 transposons. In some embodiments, the nucleic acid comprises at least 500 transposons. In some embodiments, the transposon sequence is not present in the sequence of the sample nucleic acid prior to indexing. In some embodiments, the nucleic acid is at least 5 kb in length. In some embodiments, the nucleic acid is at least 25 kb in length. In some embodiments, the nucleic acid is at least 50 kb in length. In some embodiments, the nucleic acid is at least 1 Mb in length. In some embodiments, the nucleic acid comprises RNA. In some embodiments, the nucleic acid comprises mRNA. In some embodiments, the nucleic acid comprises genomic DNA. In some embodiments, the nucleic acid comprises fetal DNA. In some embodiments, the nucleic acid comprises DNA or RNA from a tumor. In some embodiments, the nucleic acid comprises genes encoding a human immunoglobulin. In some embodiments, the nucleic acid comprises genes encoding a human T cell receptor. In some embodiments, the nucleic acid comprises genes encoding the human leukocyte antigen region. In some embodiments, the nucleic acid comprises circulating nucleic acids. In some embodiments, the nucleic acid comprises cell-free circulating nucleic acids. In some embodiments, the nucleic acid comprises viral DNA or RNA. In some embodiments, the nucleic acid comprises microbial DNA or RNA. In some embodiments, the nucleic acid comprises a portion having at least one transposon nucleic acid segment for every 5000 bases. In some embodiments, the nucleic acid comprises a portion having at least one transposon nucleic acid segment for every 1000 bases. In some embodiments, the nucleic acid comprises a portion having at least one transposon nucleic acid segment for every 500 bases.

In some aspects, an indexed nucleic acid library comprises: at least 1000 nucleic acids, wherein each of the at least 1000 nucleic acids comprises: a concatamer nucleic acid of sample nucleic acid interrupted by at least one transposon nucleic acid, wherein at least some of the transposon nucleic acid comprises: a first region comprising a transposase binding border common to a transposon library; a second region of at least 5 bases, said second region varying among said at least some transposon nucleic acid molecules; a third region common to the transposon library, wherein the third region has a length sufficient for annealing and extension of a nucleic acid primer; a fourth region of at least 5 bases, said fourth region varying among said at least some transposon nucleic acid molecules; and a fifth region comprising a transposase binding border common to the transposon library, and wherein for the transposon nucleic acid of the library, the second region and the fourth region comprise nucleic acids having sequences such that determining a second region sequence and determining a fourth region sequence allows one to assign a sequence read comprising second region sequence and a sequence read comprising fourth region sequence to a common molecule. In some embodiments, the library comprises at least 5000 nucleic acids. In some embodiments, the library comprises at least 10,000 nucleic acids. In some embodiments, the library comprises at least 50,000 nucleic acids. In some embodiments, the sample originates from a diploid organism. In some embodiments, the sample originates from a polyploid organism. In some embodiments, the sample originates from a mammal. In some embodiments, the sample originates from a plant. In some embodiments, the sample originates from a human.

In some aspects, an indexed nucleic acid primer complex comprising: a concatamer nucleic acid of sample nucleic acid interrupted by at least one transposon nucleic acid, wherein at least some of the transposon nucleic acid comprises: a first region comprising a transposase binding border common to a transposon library; a second region of at least 5 bases, said second region varying among said at least some transposon nucleic acid molecules; a third region common to the transposon library, wherein the third region has a length sufficient for annealing and extension of a nucleic acid primer; a fourth region of at least 5 bases, said fourth region varying among said at least some transposon nucleic acid molecules; and a fifth region comprising a transposase binding border common to the transposon library, and wherein for the transposon nucleic acid of the library, the second region and the fourth region comprise nucleic acids having sequences such that determining a second region sequence and determining a fourth region sequence allows one to assign a sequence read comprising second region sequence and a sequence read comprising fourth region sequence to a common molecule; and a first primer annealed to a first transposon nucleic acid, and a second primer annealed to a second transposon nucleic acid, such that after primer extension, the resulting nucleic acid extension product comprises a portion of the sample nucleic acid flanked by the fourth region of the first transposon nucleic acid and the second region of the second transposon nucleic acid.

In some aspects, an indexed sequencing library comprising: at least 1000 nucleic acids, wherein each of the at least 1000 nucleic acids comprises a portion of a sample nucleic acid from a sample, a first region and a second region, wherein the portion of the sample nucleic acid is flanked by the first region and the second region, wherein at least two nucleic acids in the library comprise a nucleic acid index pair, comprising the second region of a first nucleic acid and the first region of a second nucleic acid, wherein the portion of the sample nucleic acid in the first nucleic acid and the portion of the sample nucleic acid in the second nucleic acid are adjacent in the sample. In some embodiments, the library comprises at least 5,000 unique nucleic acids. In some embodiments, the library comprises at least 10,000 unique nucleic acids. In some embodiments, the library comprises at least 100,000 unique nucleic acids. In some embodiments, the library comprises at least 1,000,000 unique nucleic acids.

In some embodiments, a method of synthesizing the nucleic acid of any one of preceding embodiments comprises selecting a predetermined sequence for the nucleic acid; synthesizing the nucleic acid on a solid support; cleaving the nucleic acid from the solid support; and optionally amplifying the nucleic acid.

In some embodiments, a method of synthesizing the polynucleotide library of any one of the preceding embodiments comprises selecting predetermined sequences for each of the nucleic acids; synthesizing each nucleic acid on a solid support; cleaving at least some of the nucleic acids from the solid support to generate the polynucleotide library; and optionally amplifying the polynucleotide library.

In some embodiments, a method of synthesizing the sequencing library of any one of the preceding embodiments comprises contacting a sample nucleic acid interrupted by at least one transposon with two or more nucleic acid primers and a polymerase; annealing at least some of the nucleic acid primers to at least one transposon; and extending the nucleic acid primers using the sample nucleic acid interrupted by at least one transposon as a template to generate the sequencing library.

In some embodiments, a method of synthesizing the sequencing library of any one of the preceding embodiments comprises contacting a sample nucleic acid interrupted by at least one transposon with an endonuclease to form nucleic acid fragments, wherein the endonuclease is a restriction enzyme, a zinc finger nuclease (ZFN), a transcription activator-like effector nucleases (TALEN), or a CRISPR-Cas9 endonuclease; ligating a polynucleotide having a common sequence to at least some of the nucleic acid fragments to generate the sequencing library; and optionally amplifying the sequencing library.

In some embodiments, a method of sequencing a nucleic acid sample comprises: a) inserting a library of transposon nucleic acids of predetermined sequence into the nucleic acid sample; b) generating nucleic acid fragments from the nucleic acid sample, wherein at least some of the fragments comprise a portion of a transpon nucleic acid; c) sequencing the nucleic acid fragments; d) determining the sequence of the nucleic acid sample by aligning adjacent fragments that each comprise a portion of the same transposon nucleic acid.

In some aspects, a method of haplotyping a nucleic acid sample comprises: a) inserting a library of transposon nucleic acids of predetermined sequence into the nucleic acid sample; b) generating nucleic acid fragments from the nucleic acid sample, wherein at least some of the fragments comprise a portion of a transpon nucleic acid; c) sequencing the nucleic acid fragments; d) determining the sequence of the nucleic acid sample by aligning a first fragment of a first chromosome of a chromosome pair with a second fragment of the first chromosome, wherein at least one of the first fragment and the second fragment comprises a mutation that is not present in a second chromosome of the pair.

In some embodiments, a kit comprises a) a transposon library of any one of the preceding claims; b) instructions for using the transposon library; and c) optionally a transposase.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A illustrates an exemplary workflow for sample preparation, sequencing, and assembly of a nucleic acid sample using the methods described herein.

FIG. 1B illustrates an exemplary transposon polynucleotide for use in indexing a nucleic acid sample.

FIG. 1C illustrates an exemplary transposome for use in indexing a nucleic acid sample.

FIG. 2A illustrates an exemplary workflow comprising insertion/indexing of barcode sequences into the nucleic acid sample, followed by amplification of inter-barcode regions to form an amplicon library.

FIG. 2B illustrates an exemplary workflow comprising paired-end sequencing of the amplicon library, followed by in-silico assembly of sequencing reads.

FIG. 2C illustrates matching of a barcode pair from read pair data that identifies that two fragments were adjacent in a nucleic acid sample.

FIG. 3 illustrates matching of barcode pairs from read pair data that identifies that multiple fragments were adjacent in a nucleic acid sample.

FIG. 4A illustrates a sequence assembly situation wherein overlapping read pairs are insufficient for haplotyping.

FIG. 4B illustrates a sequence assembly situation wherein barcoded read pairs allow successful haplotyping.

### DETAILED DESCRIPTION OF THE INVENTION

Provided herein are methods and compositions relating to the generation of long range sequence assembly information from short read sequencers. Practice of some methods and use of some compositions herein facilitates assembly of concurrently or separately generated contig information into scaffolds up to and including chromosome-scale or genome-scale scaffolds, accurately phased, from a short read sequencing platform such as a sequencing by synthesis technology platform.

Many current nucleic acid sequence assembly approaches variously involve fragmenting a sample, attaching adapters to ends of fragments so as to facilitate sequencing by synthesis of the fragments, and generation of sequence reads from the adapters. Reads that are known to arise from a common fragment are assigned to a common ‘read pair’. Read pairs are then mapped to concurrently or independently generated sequence information, such as contigs of assembled sequence reads. When read pairs uniquely map to two separate contigs, one may confidently assign those contigs to a common phase of a common scaffold, even if the contigs do not share overlapping sequence.

A challenge of these approaches is that many read pairs do not uniquely map to a single contig, either due to their mapping to a repetitive nucleic acid segment such as a mobile element or di-, tri-, or higher order nucleic acid repeat region, or due to their mapping to a region of high homozygosity. Accordingly, polymorphisms that differ between otherwise identical alleles at a locus and that are separated by long regions of homozygosity are often not accurately assigned to a correct phase relative to one another using currently available technology. Similarly, contigs of high diversity sequence are not easily assigned to a common phase if the nucleic acids they represent are separated from one another by long stretches of repetitive sequence such as mobile element or repeat sequence.

The disclosure herein substantially reduces these issues by providing phase-preserving information in addition to read pair information, such that a given sequence read is phased not only with its read pair but also to the read representing sequence information adjacent to that read on a sample molecule. The adjacent read information is adjacent but upstream or in the direction opposite that of the other read of a read pair, such that in addition to pairing a read of a library molecule with the read pair from the opposite end of the library molecule, one may also pair the read to a read of the library molecule that arose from a nucleic acid immediately adjacent on the sample molecule to be phased. Through this approach, in some cases a plurality of read pairs are assigned to a common phase independent of whether they map to unique contig sequence, and dimorphisms in contig sequences are accurately identified despite being separated in some cases by long stretches of a sample nucleic acid that do not differ among sample molecules.

This phase information is preserved through the tagging of library constituent borders using sequence tags that allow one to identify the library constituent arising from an adjacent segment of a nucleic acid molecule. A number of approaches are disclosed to effect such tagging, such as library generation via insertion of tagging molecules having a bipartite structure comprising two tag regions that are identifiable as arising from a common insert, such that upon effecting cleavage of the insert, the fragments are identifiable as arising from a common insert at a common insert position. In some cases the two tags are an identical repeat that occurs rarely or uniquely in an insertion library, such that identification of the tag on reads of two library constituents definitively identifies the reads as arising from fragments adjacent on a common sample molecule. In some cases assigning the reads to adjacent positions on a sample molecule is facilitated by analysis of the library molecule adjacent to the tag, while in alternate cases the reads are identified as arising from adjacent segments of a sample molecule through analysis of their tags alone. Alternate tags suitable for the methods and compositions herein comprise sequence that is not identical but that shares sufficient sequence information so as to reliably assign reads having the tag sequence to adjacent positions on a sample molecule. Examples include tags that differ by a single base or a plurality of bases, but that differ such that they are readily assigned to a common insert source. Alternately, some tags share no inherent similarities, but are assigned to a common insert of origin because the sequences of inserts are known and tags can be uniquely mapped to a single insert or a sufficiently small set of inserts that adjacent read information is reliably assigned to a common phase when the tags known to co-occur on an insert are seen on reads of separate read pairs.

Tags are in some cases unique, while in other cases tags are not unique but are nonetheless rare enough so as to assign reads sharing paired tags to adjacent library constituents, such as by also considering library constituent sequence.

A consequence of the practice of the methods herein is that read pairs are confidently assigned to a common phase with other read pairs of a library independent of whether they map to a common contig, such as a contig that exhibits polymorphisms to which reads of separate read pairs map. That is, a population of read pairs is confidently assigned to a common phase, and more particularly a common order and orientation in a phased scaffold even if there are no polymorphisms indicative of phase in the reads of the read pairs or, if the read pairs are mapped to contigs, even if there are no polymorphisms indicative of phase in the contigs to which the read pairs map. That is, read pairs are ordered and oriented independent of whether they map, uniquely or otherwise, to contigs. This is accomplished through the use of insertion tags, alone or in combination with read pair sequence indicative of library constituent identity, to accurately assign reads to adjacent positions on a sample molecule through their sharing tag sequence indicative of their phase and proximity.

Accordingly disclosed herein are methods and compositions for the assembly of short sequencing reads using indexed nucleic acids to preserve proximity data. Also provided herein are methods and compositions comprising libraries of transposons to index sample nucleic acids, as well as methods and compositions for accurate genomic haplotyping. Also provided herein are methods and compositions for accurate SNP base calling.

Several commonly used NGS platforms produce short read sequences from insert sizes of several hundreds of bases. Direct sequencing of longer DNA fragments, which is often needed to obtain haplotype information, can be challenging on these platforms. Although other long sequencing techniques exist commercially (for example pore-based platforms), their accuracies and read throughput are often not as high as the short-read sequencing techniques. Several methods have been demonstrated to construct long sequences from short reads, and such methods often utilize some kind of physical partitioning of single molecules, followed by introduction of barcodes to each partition. The original single molecule in the partition is fragmented, and the same barcode sequence is appended to each fragment. Short read sequences with the same barcode may be used to reconstruct the sequence of the original single molecule.

Provided herein are compositions and methods for sequencing of nucleic acid samples using in-silico assembly of short read sequences. Such assembly methods are facilitated by indexing (barcode) information embedded into the original nucleic acid sequence by transposon libraries, which provides proximity data for establishing the original connectivity of read pairs. Single molecules are not partitioned in some instances of the methods described herein.

Provided herein are methods of sequencing nucleic acid samples. Nucleic acid samples variously comprise any sample comprising nucleic acids from naturally occurring or artificial sources, such as human, animal, plant, bacterial (eubacterial or archaeal), viral, or synthetic origin. In some cases, the nucleic acid sample comprises genomic DNA, or other source of DNA. Some samples comprise RNA, such as mRNA transcripts from a population of cells, extracellular RNA or even a single cell transcriptome, among other RNA source in some instances. In some cases samples comprise long RNA transcripts, such as transcripts at least 200, 300, 500, 1000, 2000, 3000 or more than 3000 bases in length. Optionally, nucleic acid samples comprise fetal DNA, such as ffDNA or other type of fetal DNA. Often nucleic acid samples originate from disease-related tissues or samples, such as a tumor or other disease-related sample. Specific regions of interest in a genomic sample in some instances comprise the leukocyte antigen region, a T-cell receptor, immunoglobin or other gene.

Nucleic acids from a number of sources are compatible with the methods described herein. The average size of (sample) nucleic acids often ranges from less than 100 bases to 1000 bases or more in a smaller fragment to millions of bases or billions of bases in a genomic sample or a collection of multiple genomic samples. Various nucleic acid lengths are compatible with the methods described herein. Often nucleic acids of a desired size are generated prior to contact with transposomes described herein. In some instances sample nucleic acids are at least 1000, 2000, 3000, 5000, 10,000, 100,000 or at least 1,000,000 bases in length.

### Transposons

Nucleic acid samples are assigned proximity data by application of the methods and compositions described herein. For example, a transposon library comprising transposons (nucleic acids) provides proximity data, when associated with fragments of a sample nucleic acid. Transposons often comprise one or mosaic sequences, one or more barcode sequences, a reverse primer site, and a forward primer site. Various arrangements of these elements are applied in the compositions described herein. In some instances, transposons comprise a mosaic sequence on the 5′ and 3′ ends of a polynucleotide. Such mosaic sequences are often transposase binding borders or other sequence that facilitates insertion of adjacent DNA into a nucleic acid sample. Mosaic sequences in some instances comprise elements of secondary structure, and support binding of one or more transposases. In some cases, barcode sequences are present between a mosaic sequence and an internal reverse or forward primer binding site. In some instances, a forward primer binding site and a reverse primer binding site are adjacent. Alternately, a forward primer binding site and a reverse primer binding site may overlap.

Transposons are often synthesized using either solution or solid phase-based oligonucleotide synthesis chemistry. In some cases, solid phase-based synthesis comprises synthesis on beads, columns, or chips.

Transposons often comprise one or more regions, such as regions corresponding to transposase binding borders, barcode sequences, and/or primer binding sites. In an exemplary arrangement, a transposon comprises a first region comprising a transposase binding border common to the transposon library, a second region varying among members of a transposon library, a third region common to a transposon library, wherein the third region has a length sufficient for annealing and extension of a nucleic acid primer; a fourth region varying among members of a transposon library, and a fifth region comprising a transpose binding border common to a transposon library. In some cases, the second region and the fourth region comprise nucleic acids having sequences such that determining a second region sequence and determining a fourth region sequence allows one to assign a sequence read comprising second region sequence and a sequence read comprising fourth region sequence to a common molecule. In some instances, the second region and/or the fourth region comprise at least 5 bases. In some cases, the second and/or fourth region comprises an index or barcode sequence.

Nucleic acid samples are conveyed proximity data by application of the methods and compositions described herein. Such proximity data variously comprises index sequences, barcodes, or other identifiable moiety comprising information that is used to establish proximity relationships between fragments of nucleic acids. For example, a barcode sequence comprises at least 6, 7, 8, 9, 10, 11, 12, 13, 14, 15, 20 or at least 25 bases in length. In some instances, a barcode sequence comprises at least 7 bases in length. Various barcode sequences are 5-15 bases, 7-20 bases, 8-15 bases, 10-20 bases, 9-12 bases, or 5-10 bases in length, or fall in a range comparable to those above. In some instances, a barcode sequence comprises about 8 bases. A transposon optionally comprises one or more unique barcodes relative to other members in a transposon library. Such arrangements nominally provide large numbers of unique barcode pairs, when present on the same transposon. In an exemplary arrangement, at least 5000, 7000, 10,000, 20,000, 30,000, 50,000, 100,000 or more than 100,000 pairs are present in a transposon library. In some instances, a transposon library comprises a sufficient number of barcodes to generate at least 10,000 unique pairs. In some instances, a transposon library comprises a sufficient number of barcodes to generate at least 5000, 7000, 10,000, 20,000, 30,000 or more than 30,000 unique pairs. In some cases, a portion of the barcodes differ by a single base, or differ by about 2 bases, about 3 bases, or about 4 bases. Additional nucleic acid tags, barcodes, or index sequences that provide proximity information are also consistent with the methods described herein.

Transposons described herein often comprise one or more primer binding sites, such as reverse and forward primer binding sites. The length of these primer binding sites is often varied depending on specific conditions and base identity. For example, primer binding sites are 10-30 bases, 15-25 base, 18-23 bases, or 15-30 bases in length. In some instances, primer binding sites are at least 10, 15, 20, 25, 30, 35 or at least 40 bases in length. In some cases, primer binding sites are about 20 bases in length. Primer binding site length and sequence identity are optionally varied depending on the amplification conditions and desired application.

TABLE 1

| SEQ ID NO | Name | Sequence |
|---|---|---|
| 1 | P5 | AATGATACGGCGACCACCGA |
| | | (SEQ ID NO: 1) |
| 2 | P7 | CAAGCAGAAGACGGCATACGAGAT |
| | | (SEQ ID NO: 2) |
| 3 | P1 | CCTCTCTATGGGCAGTCGGTGAT |
| | | (SEQ ID NO: 3) |

Transposons bind to transposases via mosaic sequences present on a transposon described herein. In some instances, mosaic sequences are recognized by a transposase, such as Tn5 or other transposase. In an exemplary arrangement, mosaic sequences comprise inverted repeats or other secondary structure element that facilitates transposase binding or recognition. Any number of different transposases are compatible with the methods and compositions described herein, including retrotransposases. Examples of transposases include but are not limited to DDE transposases, tyrosine transposases (e.g., Kangaroo, Tn916, and DIRS1), serine transposases (e.g., Tn5397, IS607), rolling circle/Y2 transposases (e.g., IS91, helitrons), reverse transcriptases/endonucleases (e.g., LINE-1, TP-retrotransposons), or other transposase. In some instances, a DDE transposase includes Drosphilia P element, bacteriophage Mu, Tn5 or Tn10, Mariner, IS10, and IS50. In some cases, transposases do not (or minimally) fragment the nucleic acid sample. In some instances, transposases fragment the nucleic acid sample. Often, transposases used herein insert known barcode pairs into sample nucleic acids, wherein the location of the transposon insertion can be ascertained after fragmentation and sequencing of the fragments. Other families of transposases, or transposon mutants capable of inserting transposons into nucleic acid samples are also utilized with methods and compositions herein.

### Nucleic Acid Sequencing Methods

Provided herein are methods comprising inserting transposon sequences into sample nucleic acids (such as genomic DNA, RNA, or other nucleic acid), generating nucleic acid fragments, sequencing the fragments, and matching barcodes embedded in transposon sequences to reassemble the order of the fragments.

Transposons described herein are synthesized, with each transposon comprising at least one barcode. Optionally, transposon libraries are amplified before use with primers hybridizing to mosaic sequences. In some cases, two barcodes are present on each transposon, along with recognition sequences for a corresponding transposase. Transposons are contacted with transposases to form transposomes, which are then contacted with a sample nucleic acid. The transposon sequences are inserted into positions in the nucleic acid at various positions. The locations of insertions are in some cases random, or controlled by the selection of conditions, transposase, or other factor that influences transposon insertion. Conditions include but are not limited to time, temperature, concentration, buffers, or other condition which influences transposon insertion. In some cases, addition of transposases results in minimal fragmentation of the nucleic acid, such as less than 5%, 3%, 1%, 0.1%, or less than 0.01%. In one example, two or more transposomes comprising different transposases are contacted with the nucleic acid, wherein each transposase inserts a transposon from its corresponding library into different positions of the nucleic acid. Optionally, different libraries comprise unique primer regions.

Large libraries of transposons are often contacted with sample nucleic acids, allowing for various numbers of insertion events. Optionally, the transposome to target ratio is altered to control the number or frequency of transposition events. In some instances, at least 100, 200, 500, 800, 1000, 2000, 5000, 8000, 10,000, 20,000, 50,000, 80,000, or at least 100,000 unique transposons are inserted into the sample nucleic acid. In some cases, 100-5000, 1000-10,000, 10,000-100,000, 100-1000, 50,000-100,000, or 5,000-50,000 unique transposons are inserted into the sample nucleic acid. The number of transposon events is optionally expressed in terms of the number of transposons per sample nucleic acid bases. For example, at least one transposon is inserted for every 100, 200, 300, 400, 500, 800, 1000, 2000, 5000, 8000, or 10,000 bases of the sample nucleic acid. In some instances, at least one transposon is inserted for every 100-500, 100-1000, 200-1000, 300-500, 500-1000, or 500-2000 bases of the sample nucleic acid. The number of transposons inserted into the nucleic acid often vary as a function of the size of the nucleic acid, nucleic acid source, number of unique transposons in the library, reaction conditions, or other factor.

After contact of the sample nucleic acid with a transposon library described herein, often the library is fragmented to provide nucleic acid fragments for sequencing. Various methods are used to generate such fragments, such as targeted amplification of regions comprising a portion of a first transposon, a portion of a second transposon, and a portion of the nucleic acid located between the first transposon and the second transposon to form an amplicon. Such portions of the first and second transposon in some instances comprise barcodes. Primers utilized for targeted amplification in some cases hybridize to primer binding sites on one or more transposons. In some instances, transposons comprise a universal forward and backward primer binding site. Primers often comprise additional sequences helpful for sequencing. For example, primers comprise adapter sequences, wherein adapter sequences optionally comprise graft sequences (that attach to solid surfaces, such as those on a sequencing instrument flow cell), additional barcodes (for sample identification/multiplexing, or other purpose), and sequencing primer regions. In some instances, primers comprising adapter sequences and primers without adapter sequences are employed during a single amplification reaction. Simultaneous amplification of regions of the sample nucleic acid in some cases generates an amplicon library, which is optionally sequenced. Other methods of generating sequence-ready fragments that preserve barcode information, such as endonuclease (restriction enzyme, zinc finger endonuclease, transcription activator-like effector nucleases, or CRISPR-Cas9 endonuclease, or other endonuclease) or physical shearing, are also consistent with the methods described herein. Optionally, insertion of transposons and amplification to generate fragments is performed in a single tube. In some instances, libraries of fragments are enriched prior to sequencing, such as by selective capture with labeled probes, or additional rounds of targeted PCR.

Sequencing by synthesis generates complementary strands bound to a solid support, by using members of the amplicon library (fragments) as a template strand. Bridging amplification allows generation of both forward and reverse strands of the fragments, and thus obtains a forward and backward read for a given fragment (“read pair”). In some cases, each read pair comprises sequencing data from two barcodes that originated from unique transposons. During assembly of read pairs, barcodes belonging to the same transposon are matched (linked) to assemble the fragments in the correct order and orientation in silico. This process is repeated to assemble the entire nucleic acid sample.

Sequencing data obtained from the methods and compositions described herein is useful any number of sequencing applications. For example, haplotype phasing involves assigning the correct read pair data to a specific chromosome in a diploid or polyploid organism. In some cases, nucleic acid samples originate from organisms comprising 2, 3, 4, 5, 6, 7, 8, or more than 8 sets of chromosomes. Other applications include identification of single nucleotide polymorphisms (SNPs) in a nucleic acid sample, wherein variation exists at a single position across individuals in a population. Methods described herein are often used to match variations that occur far apart on a given chromosome, for example at or near the ends of a chromosome. In some cases, haplotype matching occurs between two variants that are separated by at least 1000, 2000, 5000, 10,000, 20,000, 50,000, or more than 50,000 bases.

Methods and compositions described herein are used with a variety of different devices, such as sequencing instruments. Such instruments variously comprise technologies utilizing sequencing by synthesis, flow/pore-based technologies, pyrosequencing, ligation/probes, ion detection, or other sequencing technology. In a preferred example, the sequencing instrument generates short read pairs of 50-500 bases in length. In some cases, the sequencing instrument generates short read pairs of 100-300 bases in length.

Methods and systems described herein are often utilized for the assembly of nucleic acid sequence information. Such assembly processes variously comprises scaffold or contig assembly, scaffold or contig generation, haplotype/phasing, assembly of paired-end reads, assembly of indexed sequences that comprise proximity information, or other process that results in assembly of sequencing data.

Methods described herein are in some instances used for the assembly of sequencing data. An exemplary method for assembly comprises receiving a set of paired end reads, receiving indexing data from each paired end read, assigning paired end reads to a common phase of a scaffold, and assigning commonly indexed reads to a common phase of a scaffold. Optionally, paired-end sequencing data is used to supplement indexing data in the methods described herein. Often, methods further comprise displaying the results of an assembly on a screen, network, or server.

Computer-implemented systems are in some instances used for the assembly of sequencing data. An exemplary computer implemented system for assembly comprises a processor, wherein the processor is configured to execute the methods described herein. In an exemplary system, a processor is configured to receive a set of paired end reads, receive indexing data from each paired end read, assign paired end reads to a common phase of a scaffold, and assign commonly indexed reads to a common phase of a scaffold. Optionally, paired-end sequencing data is used to supplement indexing data in the systems described herein. Often, output generated by such a processor is displayed on a screen, network, or server.

Methods and systems described herein often have distinct advantages over other methods of sequencing, including methods that preserve proximity information in sample nucleic acid fragments. For example, methods and systems described herein in some cases reduce the amount of sample preparation time, number of reads required to assemble a sample nucleic acid, number of reads to detect an SNP, time required to phase, or other advantage. In some instances, use of transposon-based indexing results in 10% fewer reads, 20% fewer reads, 30% fewer reads, or 50% fewer reads to assemble a sample nucleic acid from sequencing data than a method that does not comprise transposon-based indexing. In some instances, use of transposon-based indexing results in 10% less time, 20% less time, 30% less time, or 50% less time to assemble a sample nucleic acid from sequencing data than a method that does not comprise transposon-based indexing.

### DISCUSSION OF THE ACCOMPANYING FIGURES

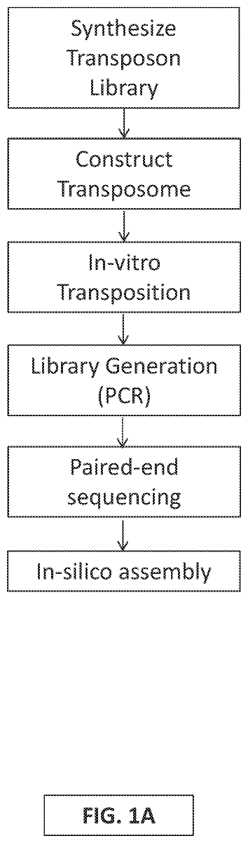

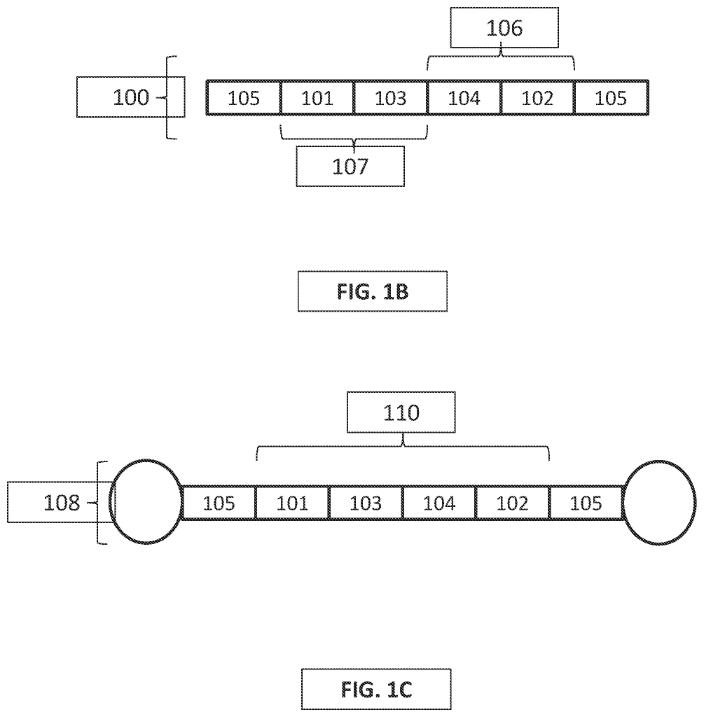

FIG. 1A illustrates an exemplary workflow for sample preparation, sequencing, and assembly of a nucleic acid sample. First, a transposon library comprise a plurality of transposons is generated, wherein transposons comprise mosaic sequences, barcode sequences, and forward/reverse primer binding sites. Second, transposases that bind to the mosaic sequences are added to form transposomes. Third, transposomes insert barcodes and primer sites into the nucleic acid sample. Fourth, inserted primer sites are used to amplify sample regions; these amplicons comprise barcode sequences, sample sequences, and adapter sequences for use in next generation sequencing. Fifth, the amplicons are sequenced; sixth, matching barcode sequences between paired end reads is used to establish the connectivity of fragments in-silico, and the original sequence is assembled. Additional steps before, after, or between the steps listed in FIG. 1A are also consistent with the method, systems, and compositions described herein.

FIG. 1B illustrates an exemplary arrangement of elements within a nucleic acid transposon 100 described herein. 5′ and 3′ mosaic sequences 105 are used to bind transposases. The pair of nucleic barcode sequences 101 and 102 preserves connectivity data after insertion into the nucleic acid sample, and fragmentation. Forward 103 and reverse 104 primer binding sites allow for PCR amplification of a portion of the transposon 107 and 106, respectively.

FIG. 1C illustrates a transposome 108, wherein transposases 108 are bound to mosaic sites 105 of the transposon 100. Such transposomes 108 are contacted with sample nucleic acids, wherein transposomes 108 insert into the sample nucleic acids. In some instances, mosaic sequences 105 also insert into the sample nucleic acid.

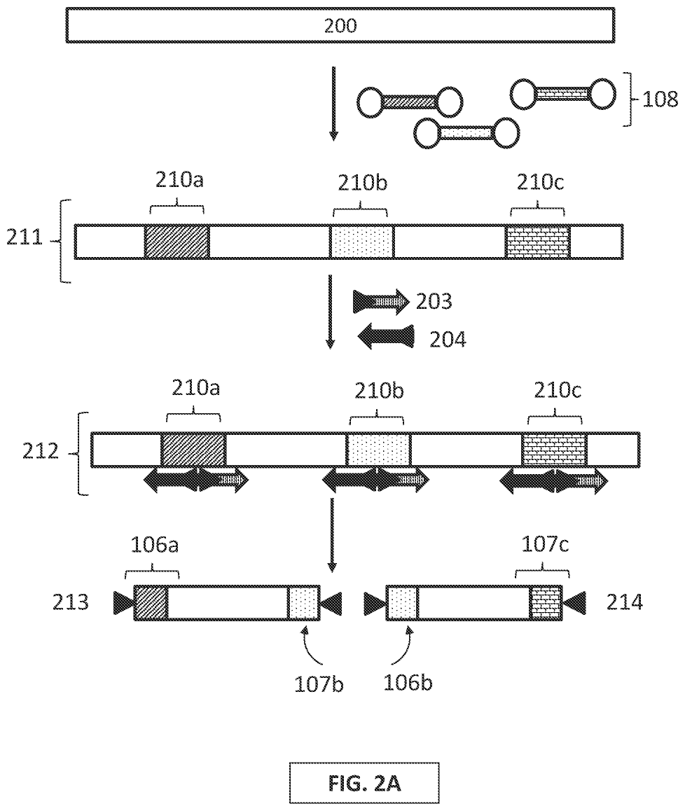

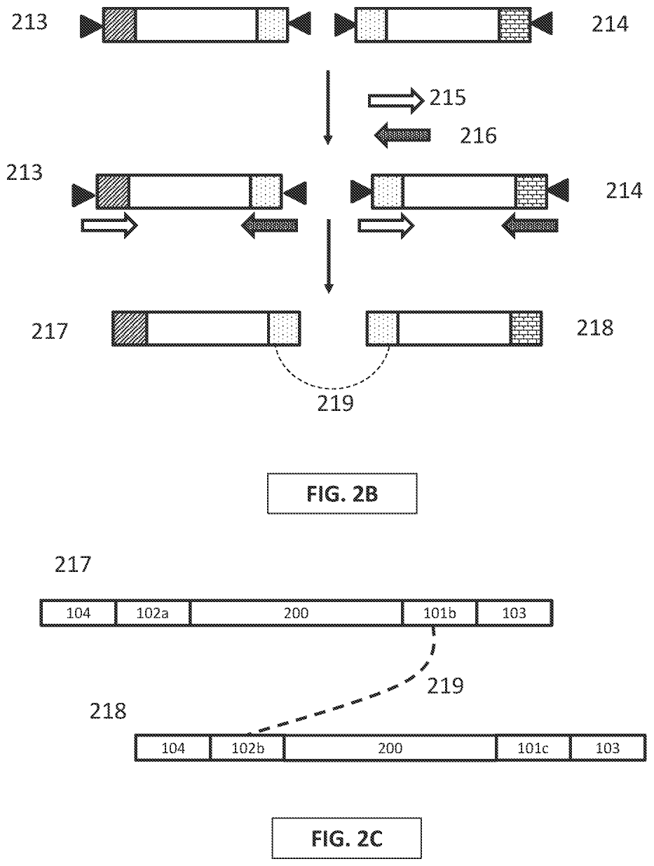

FIG. 2A illustrates an exemplary workflow for insertion of a transposome library comprising transposomes 108 into a nucleic sample to form indexed polynucleotide (transposon concatamer) 211, wherein the nucleic acid sample (sample nucleic acid) is interspersed with one or more transposons. Three transposome inserts 210a, 210b, and 210c are shown for illustration only, as a library of transposome in some cases comprises hundreds or even thousands of transposomes. After insertion, forward 203 and reverse 204 primers hybridize to primer binding sites on 210a, 210b, and 210c, to allow PCR amplification of regions between transposome insertion sites to form amplicons 213 and 214. Such amplicons comprise proximity information (barcodes) that identify their original location in the nucleic sample relative to other transposon sites. Primers 203 and 204 often comprise sequencing-instrument compatible adapter sequences, wherein amplicons 213 and 214 are immediately ready for paired-end sequencing. Proximity data is encoded into portions 106a and 107b of fragments 213 and portions 106b and 107c of transposon 214. Knowledge of the correct matching pairs (e.g., 107b/106a) allows in-silico assembly of the sequences of fragments 213 and 214, and ultimately assembly of sample nucleic acid 200.

FIG. 2B illustrates an exemplary workflow for paired-end sequencing of an amplicon library with forward 215 and reverse 216 sequencing primers, which are complementary to adapter sequences on amplicon library fragments 213 and 214. Paired end reads 217 and 218 are reassembled in-silico from matching barcode pair information, such as that exemplified by 219. Proximity data from barcode pairs from all paired end reads is then utilized to reassemble the original nucleic acid sample.

FIG. 2C illustrates sequence-detail proximity information used to join exemplary fragments 217 and 218 during in-silico assembly. Barcode sequences 101b and 102b that were present on the original transposon 210b indicate that fragments 217 and 218 were adjacent in the original nucleic acid sample. By matching of all links (similar to 219) between read pairs, the entire nucleic acid sequence is assembled. Often, fragments will still comprise mosaic sequences remaining from the transposon insertion event (not shown).

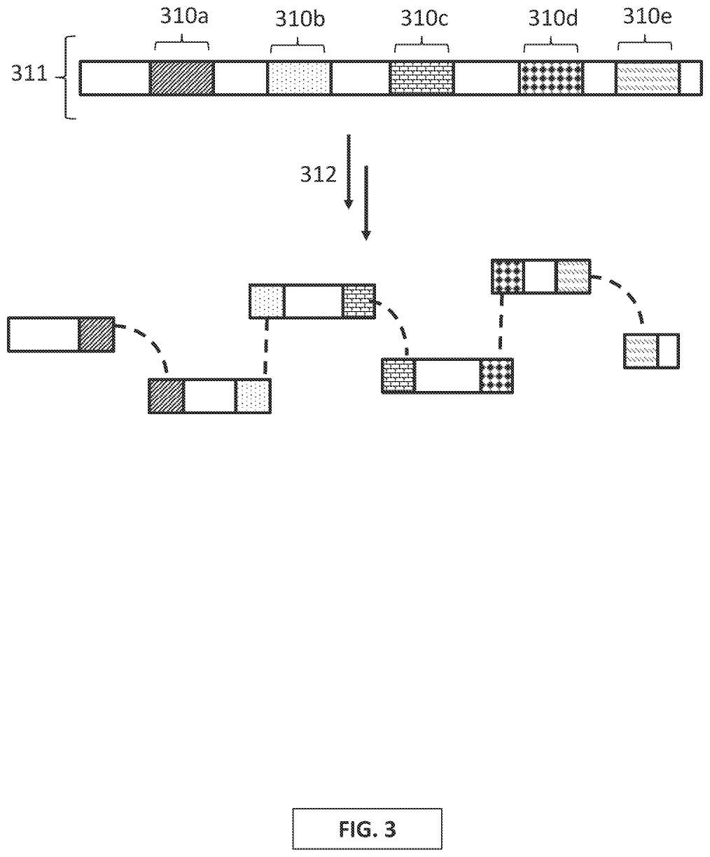

FIG. 3 illustrates an exemplary nucleic acid sample 311 comprising 5 different transposon inserts (310a-310e), wherein each transposon comprises a distinguishable barcode pair. After fragment generation and sequencing 312, read pairs are correctly matched to identify the original nucleic acid sample sequence (dotted lines). Any number of different transposons can be used in this matter to assemble read pairs.

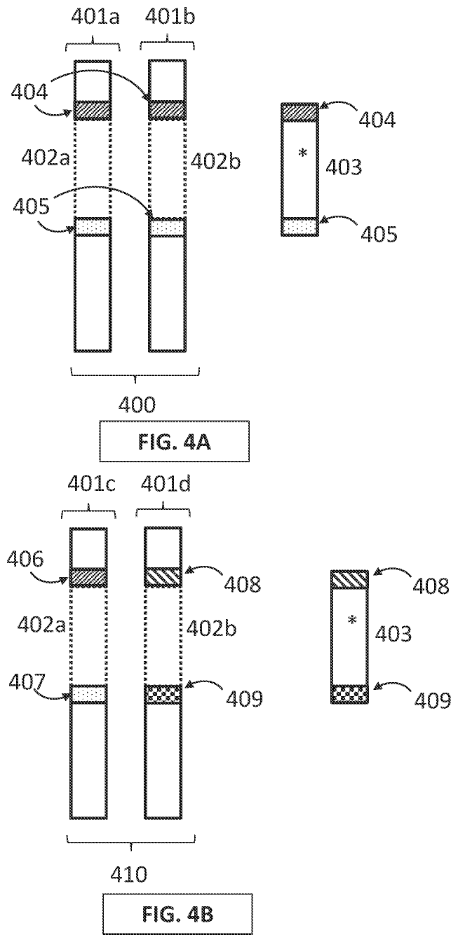

FIG. 4A illustrates an exemplary situation wherein a read pair 403 comprising a variant sequence is to be mapped to a region 402a/402b of chromosome pair 400. Corresponding overlap regions 404 and 405 of the read pair with the chromosomes are insufficient to distinguish if read pair 403 should be mapped to chromosome 401a or 402a.

FIG. 4B illustrates an exemplary situation wherein a read pair 403 comprising a variant sequence is to be mapped to a region 402a/402b of chromosome pair 410. Corresponding transposon regions 408 and 409 of the read pair uniquely identify that read pair 403 should be assigned to region 402b of chromosome 401d.

### Definitions

As used herein and in the appended claims, the singular forms “a,” “and,” and “the” include plural referents unless the context clearly dictates otherwise. Thus, for example, reference to “polynucleotide” includes a plurality of such polynucleotides and reference to “detecting a nucleotide base” includes reference to one or more methods for detecting nucleotide bases and equivalents thereof known to those skilled in the art, and so forth.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “sequencing read” as used herein, refers to a polynucleotide fragment in which the sequence has been determined. The identity of individual bases in the fragment is determined by the process of “base calling”.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

The following illustrative examples are representative of embodiments of compositions and methods described herein and are not meant to be limiting in any way.

### EXAMPLES

### Example 1: Genomic DNA Sample Sequencing and In-Silico Reassembly of Read Pairs

A researcher wishes to sequencing a genomic DNA sample using an instrument that generates short read sequences (<200 bp). The genomic DNA sample is fragmented into 200-400 bp fragments, ligated to barcode-indexed adapters, amplified, and sequenced on a commercially available sequencing instrument. The researcher is unable to reassemble portions of the genomic due to lack of substantial read pair overlap between the fragments. A large number of additional reads is required to assemble the genomic DNA sample.

### Example 2: Transposon Library-Based Indexing of a Genomic DNA Sample Prior to Sequencing

A researcher wishes to sequencing a genomic DNA sample using an instrument that generates short read sequences (<200 bp). A transposon library comprising transposons, each comprising at least 10,000 unique barcode pairs is synthesized by solid phase synthesis. Each transposon comprises two barcodes (8 bases each), a forward and a reverse primer site (each 20 bases). The library is optionally synthesized with 19 base mosaic sequences on the 5′ and 3′ ends, or alternatively mosaic sequences are added by primers during amplification of the library. Transposases are added, and the resulting transposons contacted with the genomic DNA sample. After insertion of the transposons, amplicon fragments of the indexed genomic DNA are generated using primers that bind to the transposons, wherein each primer additionally comprises an adapter sequence for next generation sequencing. After PCR, the resulting amplicon library is sequenced, and read pair data is analyzed. Barcodes from each read pair are then matched with the known matching barcode from the original transposon, and read pairs are then reassembled into the original genomic DNA sequence.

### Example 3: Haplotype Phasing

A genomic DNA sample from a human is sequenced using the general methods of Example 1. Although the sequence comprising a series of mutations in a portion of the genomic DNA sample is determined, the researcher is unable to determine which chromosome pair comprises the mutations, and is thus unable to phase the sequencing data correctly without acquiring additional data.

### Example 4: Haplotype Phasing with Transposon Library-Based Indexing

A genomic DNA sample from a human is sequenced using the general methods of Example 2. A number of read pairs have overlaps that are present in repetitive sequence regions, or are homozygous for both chromosomes of a pair. Using read pair overlap data alone, the read pairs cannot be correctly mapped. However, the read pairs are successfully assembled by matching corresponding portions of each transposon present on the read pairs, and assigning the pairs to the correct chromosome. The researcher is able to determine which chromosome of the pair comprises the mutations, and is able to successfully phase the sequencing data without acquiring additional read data.

### Example 5: SNP Calling

A genomic DNA sample from a human is sequenced using the general methods of Example 1. The researcher is unable to conclusively identify a series of single nucleotide polymorphisms (SNPs) in a portion of the genome, due to low sensitivity. The researcher must acquire large amounts of additional data until the portion of the genome has sufficient coverage to identify the SNPs.

### Example 6: SNP Calling with Transposon Library-Based Indexing

A genomic DNA sample from a human is sequenced using the general methods of Example 2. The researcher is able to conclusively identify a series of single nucleotide polymorphisms (SNPs) in a portion of the genome, due to an increased number of read pairs correctly assigned to this region.

### Example 7: Transposon Library-Based Indexing of an RNA Sample Prior to Sequencing

The general methods of Example 2 are executed with modification: the nucleic acid sample is a whole mRNA transcriptome. The transcripts are assembled using fewer total reads relative to a method that does not utilize transposon indexing of the mRNA prior to sequencing.

### Example 8: SNV Phasing with Transposon Library-Based Indexing

A genomic DNA sample from a human is sequenced using the general methods of Example 2. The researcher is able to conclusively identify a series of [C] nucleotide variants (CNVs) in a portion of the genome, due to an increased number of read pairs correctly assigned to this region.

1.-84. (canceled)

85. A method for assembling a nucleic acid scaffold, the method comprising:
a) processing a nucleic acid with a transposon library, thereby providing a transposon-processed nucleic acid, the transposon library comprising a plurality of transposon nucleic acid molecules, the plurality of transposon nucleic acid molecules comprising, in order:
a first sequence region comprising a first transposase binding border common to the transposon library;
a second sequence region, the second sequence region varying among the plurality of transposon nucleic acid molecules;
a third sequence region, the third sequence region varying among the plurality of transposon nucleic acid molecules; and
a fourth sequence region comprising a second transpose binding border common to the transposon library;
b) generating nucleic acid fragments from the transposon-processed nucleic acid, wherein the transposon-processed nucleic acid comprises a sequence corresponding to a transposon nucleic acid molecule of the plurality of transposon nucleic acid molecules;
c) sequencing at least a portion of at least some of the nucleic acid fragments to obtain a plurality of nucleic acid sequence reads; and
d) assembling the nucleic acid scaffold using nucleic acid sequencing reads of the plurality of nucleic acid sequencing reads sharing: (i) a common first sequence segment corresponding to said second sequence region and (ii) a common second sequence segment corresponding to said third sequence region.

86. The method of claim 85, wherein the second sequence region comprises at least 5 bases.

87. The method of claim 86, wherein the third sequence region comprises at least 5 bases.

88. The method of claim 85, wherein a) and b) are conducted in a single vessel.

89. The method of claim 85, wherein the nucleic acid originates from a diploid organism.

90. The method of claim 85, wherein the nucleic acid originates from a polyploid organism.

91. The method of claim 85, wherein the nucleic acid scaffold comprises at least one single nucleotide polymorphism.

92. The method of claim 85, wherein the plurality of transposon nucleic acid molecules comprise, between the second sequence region and the third sequence region, a fifth sequence region common to the transposon library, and wherein the fifth sequence region has a length sufficient for primer extension.

93. The method of claim 92, wherein b) comprises: (i) annealing a primer to a portion of said sequence corresponding to the fifth sequence region; and (ii) extending the primer via action of a polymerase.

94. The method of claim 85, wherein b) comprises contacting the transposon-processed nucleic acid with a sequence specific endonuclease.

95. The method of claim 94, wherein the endonuclease is a restriction enzyme, a zinc finger nuclease (ZFN), a transcription activator-like effector nucleases (TALEN), or a CRISPR-Cas9 endonuclease.

96. The method of claim 85, wherein b) comprises contacting the transposon-processed nucleic acid with a CRISPR-Cas9 nickase.

97. The method of claim 85, further comprising, during or after d), conducting paired-end analysis.

98. The method of claim 85, wherein the plurality of transposon nucleic acid molecules comprises at least 1000 transposon nucleic acid molecules.

99. A transposon library, comprising:
a plurality of transposon nucleic acid molecules, the plurality of transposon nucleic acid molecules having, in order:
a first sequence region comprising a transposase binding border common to the transposon library;
a second sequence region, the second sequence region varying among the plurality of transposon nucleic acid molecules;
a third sequence region common to the transposon library, wherein the third sequence region has a length sufficient for annealing and extension of a nucleic acid primer;
a fourth sequence region, the fourth sequence region varying among said plurality of transposon nucleic acid molecules; and
a fifth sequence region comprising a transposase binding border common to the transposon library.

100. The transposon library of claim 99, wherein the second sequence region and the fourth sequence region each comprise at least 5 bases.

101. The transposon library of claim 99, wherein a transposon nucleic acid molecule of the plurality of transposon nucleic acid molecules comprises a unique pair of second sequence region and fourth sequence region relative to all other members of the transposon library.

102. The transposon library of claim 99, wherein the plurality of transposon molecules comprises at least 5,000 transposon nucleic acid molecules having a unique combination of second sequence region and fourth sequence region.

103. The transposon library of claim 99, wherein a transposon nucleic acid molecule of the plurality of transposon nucleic acid molecules comprises a unique second sequence region and a unique fourth sequence region relative to all other members of the transposon library.

104. The transposon library of claim 99, wherein the transposon library comprises at least 1,000 nucleic acid transposon molecules.


SEQUENCE LISTING (3 sequences)

SEQ ID NO: 1 - DNA, 20 bp, Artificial Sequence
  Description of Artificial Sequence Synthetic primer
aatgatacggcgaccaccga

SEQ ID NO: 2 - DNA, 24 bp, Artificial Sequence
  Description of Artificial Sequence Synthetic primer
caagcagaagacggcatacgagat

SEQ ID NO: 3 - DNA, 23 bp, Artificial Sequence
  Description of Artificial Sequence Synthetic primer
cctctctatgggcagtcggtgat